(19) United States
(12) Patent Application Publication
(10) Pub. No.: US 2022/0322976 A1
(43) Pub. Date: Oct. 13, 2022

(54) FILTERING OF CONTINUOUS GLUCOSE MONITOR (CGM) SIGNALS WITH A KALMAN FILTER

(71) Applicant: DexCom, Inc., San Diego, CA (US)

(72) Inventors: Shwetha R. Edla, San Diego, CA (US); Rasoul Yousefi, San Diego, CA (US); Neda Ehtiati, San Diego, CA (US); Ghazaleh R. Esmaili, San Diego, CA (US)

(73) Assignee: Dexcom, Inc., San Diego, CA (US)

(21) Appl. No.: 17/708,892

(22) Filed: Mar. 30, 2022

Related U.S. Application Data
(60) Provisional application No. 63/168,867, filed on Mar. 31, 2021.
(60) Provisional application No. 63/208,362, filed on Jun. 8, 2021.

(51) Int. Cl. A61B 5/145; A61B 5/00

(52) U.S. Cl. CPC A61B 5/14532; A61B 5/725; A61B 5/7203

(57) ABSTRACT

In accordance with a system and/or method for monitoring an analyte concentration, a sensor signal indicative of an analyte concentration in a host may be received from an analyte sensor. The sensor signal may be filtered using a Kalman filter having process noise with a process covariance and measurement noise with a measurement covariance. The filtering may include updating a value associated with at least one of the process covariance and the measurement covariance using a value associated with one or more parameters employed in a model of the Kalman filter. A filtered sensor signal representative of the analyte concentration in the host may be output from the Kalman filter.

### RELATED APPLICATIONS

This application claims priority under 35 U.S.C. § 119(e) to U.S. Provisional Patent Application No. 63/168,867 filed Mar. 31, 2021 and titled “Filtering of CGM Signals with a Kalman Filter,” and to U.S. Provisional Patent Application No. 63/208,362 filed Jun. 8, 2021 and titled “Filtering of Continuous Glucose Monitor (CGM) Signals with a Kalman Filter”, the entire disclosures of which is hereby incorporated by reference.

### BACKGROUND

Diabetes mellitus is a disorder in which the pancreas cannot create sufficient insulin (Type I or insulin-dependent) and/or in which insulin is not effective (Type II or non-insulin-dependent). In the diabetic state, the patient or user suffers from high blood sugar, which can cause an array of physiological derangements associated with the deterioration of small blood vessels, for example, kidney failure, skin ulcers, or bleeding into the vitreous of the eye. A hypoglycemic reaction (low blood sugar) can be induced by an inadvertent overdose of insulin, or after a normal dose of insulin or glucose-lowering agent accompanied by extraordinary exercise or insufficient food intake.

Conventionally, a person with diabetes carries a self-monitoring blood glucose (SMBG) monitor, which typically requires uncomfortable finger pricking methods. Due to the lack of comfort and convenience, a person with diabetes normally only measures his or her glucose levels two to four times per day. Unfortunately, such time intervals are so far spread apart that the person with diabetes likely finds out too late of a hyperglycemic or hypoglycemic condition, sometimes incurring dangerous side effects. It is not only unlikely that a person with diabetes will become aware of a dangerous condition in time to counteract it, but it is also likely that he or she will not know whether his or her blood glucose concentration value is going up (higher) or down (lower) based on conventional methods. Diabetics thus may be inhibited from making educated insulin therapy decisions.

Another device that some diabetics used to monitor their blood glucose is a continuous analyte sensor, e.g., a continuous glucose monitor (CGM) system. A CGM system typically includes a sensor that is placed invasively, minimally invasively, or non-invasively. The sensor measures the concentration of a given analyte within the body, e.g., glucose, and generates a raw signal using electronics associated with the sensor. The raw signal is converted into an output value that is rendered on a display. The output value that results from the conversion of the raw signal is typically expressed in a form that provides the user with meaningful information, and in which form users have become familiar with analyzing, such as blood glucose expressed in mg/dL

Some CGM systems rely upon a blood glucose (BG) fingerstick meter value to correlate the sensor signal to clinical blood glucose, while others do not require real time BG fingerstick meter values to correlate (calibrate/transform) the sensor-derived raw signal into a clinical blood glucose equivalent value representative of the glucose concentration in a patient (e.g., based instead on factory information). Both types of systems may suffer from inaccuracies, particularly near the beginning or end of the sensor's life, which may result from BG values or calibration codes being interpreted too simplistically.

### SUMMARY

In a first aspect, a method is provided for monitoring a blood analyte concentration in a host, comprising: receiving from a continuous analyte sensor a sensor signal indicative of a blood analyte concentration in a host; filtering the sensor signal using a Kalman filter having process noise with a process covariance and measurement noise with a measurement covariance, wherein the filtering includes updating a value of at least one of the process covariance and the measurement covariance using a value of one or more parameters employed in a model of the Kalman filter; and outputting from the Kalman filter a filtered sensor signal representative of the blood analyte concentration in the host.

In an embodiment of the first aspect, the one or more parameters used to update at least one of the process covariance and the measurement covariance includes a value of an innovation term and a residual term employed in the Kalman filter model.

In an embodiment of the first aspect, the updating is performed when one or more predefined artifacts are detected in the sensor signal.

In an embodiment of the first aspect, the updating is performed when one or more predefined artifacts are detected in the sensor signal after filtering the sensor signal using the Kalman filter.

In an embodiment of the first aspect, the method further comprises detecting the one or more predefined artifacts by examining a residual signal, the residual signal being a difference between the sensor signal received from the analyte sensor and the sensor signal after filtering the sensor signal using the Kalman filter.

In an embodiment of the first aspect, the residual signal is a temporary residual signal that is a difference between the sensor signal received from the analyte sensor and the sensor signal after filtering the sensor signal using the filter before the at least one of the process covariance and the measurement covariance is updated.

In an embodiment of the first aspect, the residual signal is a final residual signal that is a difference between the sensor signal received from the analyte sensor and the sensor signal after filtering the sensor signal using the filter after the at least one of the process covariance and the measurement covariance is updated.

In an embodiment of the first aspect, one of the predefined artifacts is a value of a residual difference or a derivative of the residual difference that exceeds a threshold value, the residual difference being a difference between a value of a temporary residual signal and a value of a final residual signal, the temporary residual signal being a difference between the sensor signal received from the analyte sensor and the sensor signal after filtering the sensor signal using the filter before the at least one of the process covariance and the measurement covariance is updated and the final residual signal being a difference between the sensor signal received from the analyte sensor and the sensor signal after filtering the sensor signal using the filter after the at least one of the process covariance and the measurement covariance is updated.

In an embodiment of the first aspect, one of the predefined artifacts is a residual bias reflecting that the residual signal has a consistently positive or negative value over one or more selected windows of time.

In an embodiment of the first aspect, one of the predetermined artifacts is a zero crossing of the final residual signal, the zero crossing of the final residual signal reflecting a number of times a value of the final residual signal undergoes a change in sign from positive to negative or negative to positive over one or more selected windows of time.

In an embodiment of the first aspect, the one or more predetermined artifacts are based on models of the sensor signal.

In an embodiment of the first aspect, the method further comprises undoing a previous update to the values of at least one of the process covariance and the measurement covariance upon detecting one or more specified artifacts in the sensor signal.

In an embodiment of the first aspect, the one or more parameters used to update at least one of the process covariance and the measurement covariance includes a fault metric that is based on a value of an innovation term and an innovation covariance employed in the Kalman filter model.

In an embodiment of the first aspect, the fault metric is a moving average of an instantaneous fault metric averaged over a specified number of measurement samples received from the analyte sensor.

In an embodiment of the first aspect, the one or more predefined artifacts includes a value of a fault metric that exceeds a threshold, the fault metric being based on an innovation term and an innovation covariance employed in the Kalman filter model.

In an embodiment of the first aspect, the method further comprises adaptively performing the updating after each iteration of the filtering.

In an embodiment of the first aspect, the update is adaptively performed using a residual signal and specified step size coefficients, the residual signal being a difference between the sensor signal received from analyte sensor and the sensor signal after filtering the sensor signal using the Kalman filter.

In an embodiment of the first aspect, the specified step size coefficients are adjusted using transfer functions that are based on the fault metric.

In an embodiment of the first aspect, the process covariance has a minimum value that is adjusted using the transfer functions.

In an embodiment of the first aspect, the method further comprises adjusting design parameters employed in the transfer functions to achieve a prescribed tradeoff between signal smoothing and time lag.

In an embodiment of the first aspect, the method further comprises performing a corrective action upon detecting one or more artifacts in the sensor signal when the sensor signal is a low-resolution signal, the corrective action being determined at least in part by a sign of a residual signal, the residual signal being a difference between the sensor signal received from the analyte sensor and the sensor signal after filtering the sensor signal using the Kalman filter.

In an embodiment of the first aspect, the method further comprises retroactively determining from historical data an optimal Kalman filter model that was previously employed when the sensor signal is a high-resolution signal.

In an embodiment of the first aspect, the determining is performed using a residual bias and a zero crossing, the residual bias reflecting that a residual signal has a consistently positive or negative value over one or more selected windows of time and the zero crossing reflecting a number of times the residual signal undergoes a change in sign from positive to negative or negative to positive over one or more selected windows of time.

In an embodiment of the first aspect, the method comprises performing a corrective action upon detecting one or more artifacts in the sensor signal, the corrective action including updating values one or more of the parameters employed in the Kalman filter model, the updated values being selected to achieve a prescribed tradeoff between an amount of analyte sensor signal smoothing to be achieved and a time lag in tracking changes in the analyte sensor signal.

In an embodiment of the first aspect, the method further comprises determining if a feature identified in the sensor signal is to be classified as a predefined artifact using a rules-based model trained using clinical data.

In an embodiment of the first aspect, the method further comprises determining if a feature identified in the sensor signal is to be classified as a predefined artifact using a machine-learning model.

In an embodiment of the first aspect, one of the predefined artifacts is a value of residual kurtosis or an R/Q value.

In an embodiment of the first aspect, the at least one of the artifacts is identified in a sensor signal domain.

In an embodiment of the first aspect, at least one of the artifacts is identified after translation of the sensor signal to a corresponding blood glucose value.

In a second aspect, a method is provided for monitoring a blood analyte concentration in a host, comprising: receiving from a continuous analyte sensor a sensor signal indicative of a blood analyte concentration in a host; filtering the sensor signal using a Kalman filter; detecting one or more predefined artifacts in the sensor signal; performing a corrective action upon detecting the one or more artifacts in the sensor signal, wherein the corrective action includes updating values one or more of parameters employed in a model of Kalman filter; and outputting from the Kalman filter a filtered sensor signal representative of the blood analyte concentration in the host.

The difference between the raw sensor signal and filtered signal by the Kalman filter is representative of the noise on the signal. This value is used to measure the signal-to-noise ratio of the signal and is indicative of the signal quality. Other metrics can be used to provide additional signal quality metrics, such as the covariance of error calculated by the Kalman filter which can be a measure of the accuracy of the state estimates.

### BRIEF DESCRIPTION OF THE DRAWINGS

The details of the present disclosure, both as to its structure and operation, may be understood in part by study of the accompanying drawings, in which like reference numerals refer to like parts. The drawings are not necessarily to scale, emphasis instead being placed upon illustrating the principles of the disclosure.

FIG. 1 is a diagram of one example of an integrated system including a continuous glucose sensor and a medicament delivery device.

FIG. 2 is a front elevation view of an electronic device configured for use with the present systems and methods.

FIG. 3 is a functional block diagram of the electronic device of FIG. 2.

FIG. 4 is a simplified block diagram showing the primary inputs to and outputs from a Kalman filter module.

FIG. 5 shows is a graph showing the glucose level of a patient over a period of time as provided by a CGM system before the raw sensor signal is filtered.

FIG. 6 shows a simplified block diagram of an example of a Kalman filter in which an artifact detection module is employed to examine a sensor signal output by a Kalman filter state update module.

FIG. 7 shows a simplified block diagram of an example of a Kalman filter in which a fault metric calculation module is employed to examine various internal variables used by a Kalman filter update module.

FIG. 8 shows the raw sensor signal shown in FIG. 5, except the signal is filtered using a Kalman filter configured in accordance with the techniques described herein.

FIG. 9. shows a raw sensor signal and a filtered sensor signal after filtering with a Kalman filter using three different sets of parameters.

FIG. 10 is a flowchart showing a method for monitoring a blood analyte concentration in a host.

### DETAILED DESCRIPTION

Exemplary embodiments disclosed herein relate to the use of a glucose sensor that measures a concentration of glucose or a substance indicative of the concentration or presence of another analyte. In some embodiments, the glucose sensor is a continuous device, for example a subcutaneous, transdermal, transcutaneous, non-invasive, intraocular and/or intravascular (e.g., intravenous) device. In some embodiments, the device is a non-continuous device. In some embodiments, the device can analyze a plurality of intermittent blood samples. The glucose sensor can use any method of glucose measurement, including enzymatic, chemical, physical, electrochemical, optical, optochemical, fluorescence-based, spectrophotometric, spectroscopic (e.g., optical absorption spectroscopy, Raman spectroscopy, etc.), polarimetric, calorimetric, iontophoretic, radiometric, and the like.

The glucose sensor can use any known detection method, including invasive, minimally invasive, and non-invasive sensing techniques, to provide a data stream indicative of the concentration of the analyte in a host. The data stream is typically a raw data signal that is used to provide a useful value of the analyte to a user, such as a patient or health care professional (e.g., doctor), who may be using the sensor.

Although much of the description and examples are drawn to an implantable glucose sensor capable of measuring the concentration of glucose in a host, the systems and methods of embodiments can be applied to any measurable analyte and/or analytes. It should be understood that the systems, devices and/or methods described herein can be applied to any system, device, and/or method capable of detecting a concentration of an analyte and providing an output signal that represents the concentration of the analyte.

As noted, in some embodiments, the analyte sensor is an implantable glucose sensor, such as described with reference to U.S. Pat. No. 6,001,067 and U.S. Patent Publication No. US-2011-0027127-A1. In some embodiments, the analyte sensor is a transcutaneous glucose sensor, such as described with reference to U.S. Patent Publication No. US-2006-0020187-A1. In yet other embodiments, the analyte sensor is a dual electrode analyte sensor, such as described with reference to U.S. Patent Publication No. US-2009-0137887-A1. In still other embodiments, the sensor is configured to be implanted in a host vessel or extracorporeally, such as is described in U.S. Patent Publication No. US-2007-0027385-A1. These patents and publications are incorporated herein by reference in their entirety.

The following description and examples describe the present embodiments with reference to the drawings. In the drawings, reference numbers label elements of the present embodiments. These reference numbers are reproduced below in connection with the discussion of the corresponding drawing features.

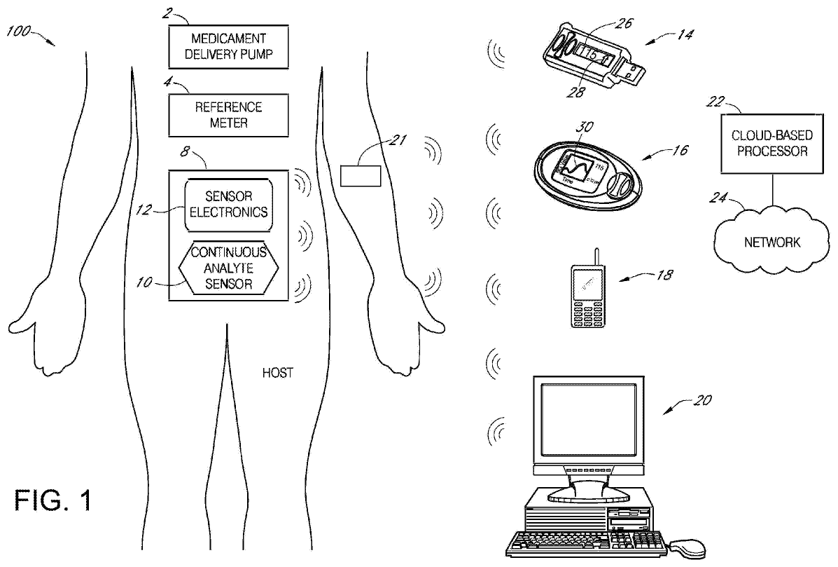

FIG. 1 is a block diagram of an integrated system of the preferred embodiments, including a continuous glucose sensor and a medicament delivery device. Such is an exemplary environment in which some embodiments described herein may be implemented. Here, an analyte monitoring system 100 includes a continuous analyte sensor system 8. Continuous analyte sensor system 8 includes a sensor electronics (e.g., a sensor electronics module) 12 and a continuous analyte sensor 10. The system 100 can also include other devices and/or sensors, such as a medicament delivery pump 2 and/or a reference analyte meter 4. The continuous analyte sensor 10 may be physically connected to sensor electronics 12. The sensor electronics 12 may be integral with (e.g., non-releasably attached to) or releasably attachable to the continuous analyte sensor 10. Alternatively, the continuous analyte sensor 10 may be physically separate from sensor electronics 12, but electronically coupled via inductive coupling or the like. Further, the sensor electronics 12, medicament delivery pump 2, and/or analyte reference meter 4, may communicate with one or more additional devices, such as any or all of display devices 14, 16, 18, and/or 20. The display devices 14, 16, 18, and 20 may generally include a processor, memory, storage, and other components sufficient to run an application including a decision support module.

As used herein, the term “continuous” used in connection with analyte monitoring may refer to an ability of a device to produce measurements substantially continuously, such that the device may be configured to produce the glucose measurements at intervals of time (e.g., every hour, every 30 minutes, every 5 minutes, and so forth). In various embodiments, however, the systems and techniques discussed herein may be implemented using non-continuous sensors and systems. For instance, the continuous analyte sensor system 8 may be implemented with a non-continuous analyte sensor which may be configured to produce analyte measurements (e.g., glucose measurements) when requested, e.g., responsive to a user request.

In some implementations, the system 100 of FIG. 1 may also include a processor (e.g., cloud-based) 22 configured to analyze analyte data, medicament delivery data and/or other user-related data provided over network 24 directly or indirectly from one or more of sensor system 8, medicament delivery pump 2, reference analyte meter 4, and/or display devices 14, 16, 18, 20. Based on the received data, the processor 22 can further process the data, generate reports providing statistics based on the processed data, trigger notifications to electronic devices associated with the host or caretaker of the host, and/or provide processed information to any of the other devices of FIG. 1. In some exemplary implementations, the processor 22 comprises one or more servers. If the processor 22 comprises multiple servers, the servers can be either geographically local or separate from one another. The network 24 can include any wired and wireless communication medium to transmit data, including WiFi networks, cellular networks, the Internet and any combinations thereof.

In some exemplary implementations, the sensor electronics 12 may include electronic circuitry associated with measuring and processing data generated by the continuous analyte sensor 10. This generated continuous analyte sensor data may also include algorithms, which can be used to process and calibrate the continuous analyte sensor data, although these algorithms may be provided in other ways as well, such as by the devices 14, 16, 18, and/or 20. The sensor electronics 12 may include hardware, firmware, software, or a combination thereof, to provide measurement of levels of the analyte via a continuous analyte sensor or a non-continuous analyte sensor (e.g., a continuous glucose sensor or a non-continuous glucose sensor).

The sensor electronics 12 may, as noted, couple (e.g., wirelessly and the like) with one or more devices, such as any or all of display devices 14, 16, 18, and 20. The display devices 14, 16, 18, and/or 20 may be configured for processing and presenting information, such sensor information transmitted by the sensor electronics module 12 for display at the display device. The display devices 14, 16, 18, and 20 can also trigger alarms and/or provide decision support recommendations based on the analyte sensor data.

In FIG. 1, display device 14 is a key fob-like display device, display device 16 is a hand-held application-specific computing device (e.g., a DexCom receiver and/or other receiver commercially available or previously commercially available from DexCom, Inc.), display device 18 is a general purpose smart phone or tablet computing device 20 (e.g., a phone running the Android™ OS, an Apple™ iPhone™, iPad™, or iPod Touch™. commercially available or previously commercially available from Apple, Inc.), and display device 20 is a computer workstation 20. In some exemplary implementations, the relatively small, key fob-like display device 14 may be a computing device embodied in a wrist watch, a belt, a necklace, a pendent, a piece of jewelry, an adhesive patch, a pager, a key fob, a plastic card (e.g., credit card), an identification (ID) card, and/or the like. This small display device 14 may include a relatively small display (e.g., smaller than the display device 18) and may be configured to display a limited set of displayable sensor information, such as a numerical value 26 and an arrow 28. Some systems may also include a wearable device 21, such as described in U.S. Provisional Patent Application No. 61/904,341, filed Nov. 14, 2013, and entitled “Devices and Methods for Continuous Analyte Monitoring,” the entire disclosure of which is hereby expressly incorporated by reference. The wearable device 21 may include any device(s) that is/are worn on, or integrated into, a user's vision, clothes, and/or bodies. Example devices include wearable devices, anklets, glasses, rings, necklaces, arm bands, pendants, belt clips, hair clips/ties, pins, cufflinks, tattoos, stickers, socks, sleeves, gloves, garments (e.g. shirts, pants, underwear, bra, etc.), “clothing jewelry” such as zipper pulls, buttons, watches, shoes, contact lenses, subcutaneous implants, eyeglasses, cochlear implants, shoe inserts, braces (mouth), braces (body), medical wrappings, sports bands (wrist band, headband), hats, bandages, hair weaves, nail polish, artificial joints/body parts, orthopedic pins/devices, implantable cardiac or neurological devices, etc. The small display device 14 and/or the wearable device 21 may include a relatively small display (e.g., smaller than the display device 18) and may be configured to display graphical and/or numerical representations of sensor information, such as a numerical value 26 and/or an arrow 28. In contrast, display devices 16, 18 and 20 may be larger display devices that may be capable of displaying a larger set of and/or different displayable information or form of displayable information, such as a trend graph 30 depicted on the hand-held receiver 16 in addition to, and/or in replacement of other information such as a numerical value and arrow.

It is understood that any other user equipment (e.g., computing devices) configured to at least present information (e.g., a medicament delivery information, discrete self-monitoring analyte readings, heart rate monitor, caloric intake monitor, and the like) may be used in addition to or instead of those discussed with reference to FIG. 1.

In some exemplary implementations of FIG. 1, the continuous analyte sensor 10 comprises a sensor for detecting and/or measuring analytes, and the continuous analyte sensor 10 may be configured to continuously detect and/or measure analytes as a non-invasive device, a subcutaneous device, a transdermal device, and/or an intravascular device. In some exemplary implementations, the continuous analyte sensor 10 may analyze a plurality of intermittent blood samples, although other analytes may be used as well. In one or more implementations, the sensor 10 may instead be implemented as a non-continuous analyte sensor.

In some exemplary implementations of FIG. 1, the continuous analyte sensor 10 may comprise a glucose sensor configured to measure glucose in the blood using one or more measurement techniques, such as enzymatic, chemical, physical, electrochemical, fluorescent, spectrophotometric, polarimetric, calorimetric, iontophoretic, radiometric, immunochemical, and the like. In implementations in which the continuous analyte sensor 10 includes a glucose sensor, the glucose sensor may be comprise any device capable of measuring the concentration of glucose and may use a variety of techniques to measure glucose including invasive, minimally invasive, and non-invasive sensing techniques (e.g., fluorescent monitoring), to provide data, such as a data stream, indicative of the concentration of glucose in a host. The data stream may be a raw data signal, which is converted into a calibrated and/or filtered data stream used to provide a value of glucose to a host, such as a user, a patient, or a caregiver (e.g., a parent, a relative, a guardian, a teacher, a doctor, a nurse, or any other individual that has an interest in the wellbeing of the host). Moreover, the continuous analyte sensor 10 may be implanted as at least one of the following types of sensors: an implantable glucose sensor, a transcutaneous glucose sensor, implanted in a host vessel or extracorporeally, a subcutaneous sensor, a refillable subcutaneous sensor, intraocular, or an intravascular sensor. As described throughout, the sensor 10 may alternately be implemented as a non-continuous glucose sensor in one or more embodiments.

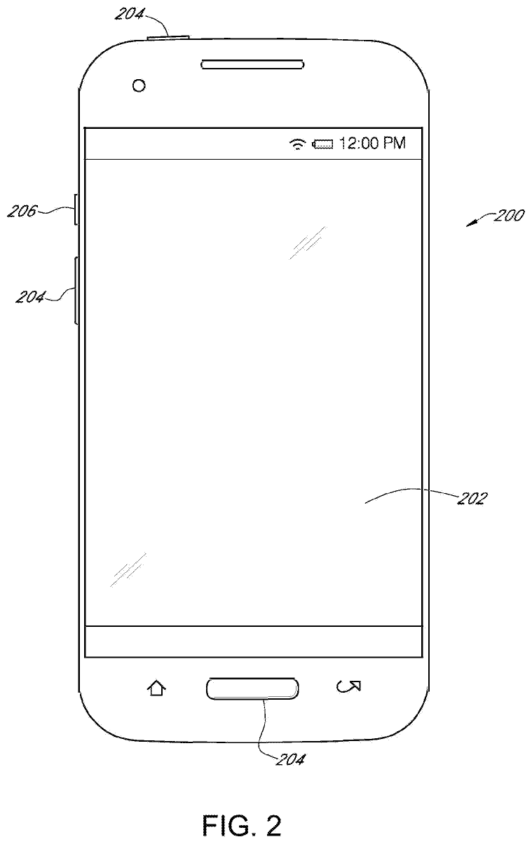

FIG. 2 illustrates one embodiment of an electronic device 200 configured for use with the present systems and methods. The electronic device 200 includes a display 202 and one or more input/output (I/O) devices, such as one or more buttons 204 and/or switches 206, which when activated (e.g., clicked and/or manipulated) perform one or more functions. In some embodiments the electronic device 200 may be mobile communication device. For instance, in the illustrated embodiment, the electronic device 200 is a smartphone, and the display 202 comprises a touchscreen, which also functions as an I/O device. In other embodiments, the electronic device 200 may comprise a device or devices other than a smartphone, such as a receiver of a CGM system, a smartwatch, a tablet computer, a mini-tablet computer, a handheld personal digital assistant (PDA), a game console, a multimedia player, a wearable device, such as those described above, a screen in an automobile or other vehicle, etc. While the electronic device 200 is illustrated as a smartphone in the figures, the electronic device 200 can be any of the other electronic devices mentioned herein and/or incorporate the functionality of any or all of the other electronic devices, including wherein some or all of the functionally is embodied on a remote server. As described in greater detail herein, in certain embodiments, processing of data such as that data discussed herein (e.g., data of a CGM system) may be performed by the electronic device 200 using one or more processors of the electronic device 200. Alternately or additionally, the processing and filtering of data discussed herein may be performed by one or more devices other than the device 200. For example, the processing and filtering techniques discussed herein may be performed, at least partially, by a wearable device (e.g., wearable device 21) that is worn on the user's body and communicates information to another device, such as the electronic device 200.

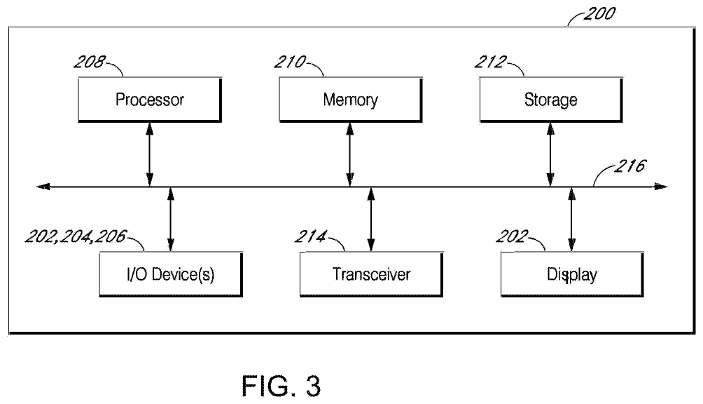

FIG. 3 is a block diagram of the electronic device 200 shown in FIG. 2, illustrating its functional components in accordance with some embodiments. The electronic device 200 includes the display 202 and one or more input/output (“I/O”) device(s) 204, 206, as described above with respect to FIG. 2. The display 202 may be any device capable of displaying output, such as an LCD or LED screen and others. The input/output (I/O) devices 202, 204, 206 may comprise, for example, a keyboard (not shown), one or more buttons 204, one or more switches 206, etc. In embodiments including a touchscreen, the display 202 also functions as an I/O device.

The electronic device 200 further includes a processor 208 (also referred to as a central processing unit (CPU)), a memory 210, a storage device 212, a transceiver 214, and may include other components or devices (not shown). The memory 210 is coupled to the processor 208 via a system bus or a local memory bus 216. The processor 208 may be, or may include, one or more programmable general-purpose or special-purpose microprocessors, digital signal processors (DSPs), programmable controllers, application specific integrated circuits (ASICs), programmable logic devices (PLDs), or the like, or a combination of such hardware-based devices.

The memory 210 provides the processor 208 access to data and program information that is stored in the memory 210 at execution time. Typically, the memory 210 includes random access memory (RAM) circuits, read-only memory (ROM), flash memory, or the like, or a combination of such devices.

The storage device 212 may comprise one or more internal and/or external mass storage devices, which may be or may include any conventional medium for storing large volumes of data in a non-volatile manner. For example, the storage device 212 may include conventional magnetic disks, optical disks, magneto-optical (MO) storage, flash-based storage devices, or any other type of non-volatile storage devices suitable for storing structured or unstructured data. The storage device 212 may also comprise storage in the “cloud” using so-called cloud computing. Cloud computing pertains to computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction.

The electronic device 200 may perform various processes, such as, for example, correlating data, pattern analysis, and other processes. In some embodiments, the electronic device 200 may perform such processes on its own. Alternatively, such processes may be performed by one or more other devices, such as one or more cloud-based processors 22 described above. In still further embodiments, these processes may be performed in part by the electronic device 200 and in part by other devices. Various example processes are described herein with reference to the electronic device 200. It should be understood that these example processes are not limited to being performed by the electronic device 200 alone. Further, as used herein, the term “electronic device” should be construed to include other devices with which the electronic device 200 interacts, such as one or more cloud-based processors, servers, etc.

The electronic device 200 may also include other devices/interfaces for performing various functions. For example, the electronic device 200 may include a camera (not shown).

The transceiver 214 enables the electronic device 200 to communicate with other computing systems, storage devices, and other devices via a network. While the illustrated embodiment includes a transceiver 214, in alternative embodiments a separate transmitter and a separate receiver may be substituted for the transceiver 214.

In some embodiments, the processor 208 may execute various applications, for example, a CGM application, which is loaded on the electronic device 200. The application (e.g., the CGM application) may be downloaded to the electronic device 200 over the Internet and/or a cellular network, and the like. Data for various applications may be shared between the electronic device 200 and one or more other devices/systems, and stored by storage 212 and/or on one or more other devices/systems. This CGM application may include a decision support electronics (e.g., a decision support module) and/or may include processing sufficient to operate decision support assessment functions and methods as described below.

In certain of the present embodiments, the sensor 10 of the continuous analyte sensor system 8 of FIG. 1 is inserted into the skin of a host. A new sensor session is initiated with the sensor 10, the sensor electronics 12, and the electronic device 200. Numerous techniques may be employed for initializing the sensor 10. For example, initialization may be triggered when the sensor electronics 12 engages the sensor 10. In another example, initialization may be triggered by a mechanical switch, such as a switch (not shown) on a snap-in base that receives the sensor electronics 12. When the sensor electronics 12 are snapped into the base, the switch is automatically tripped. In another example, initialization may be menu driven, and the user may be prompted by a user interface on the display 202 of the electronic device 200 to begin initialization by making a selection on the user interface, such as by pushing a button or touching a designated area on a display 202 (which may comprise a touchscreen). In another example, initialization may be based upon evaluation or analysis of a signal characteristic, such as a signal received by the sensor electronics 12 from the sensor 10. In another example involving a non-invasive sensor that is applied to the wearer's skin, the sensor 10 may sense when it is in contact with skin and start automatically. Further, the analyte sensor system 8 can detect use of a new sensor 10 using any of the above techniques, automatically prompt the user to confirm the new sensor session by way of a prompt on a user interface of the system 8, and initiate an initialization response to the user confirmation responsive to the prompt. Additional examples of initializing the sensor 10 are found in U.S. patent application Ser. No. 13/796,185, filed on Mar. 12, 2013, the entire disclosure of which is hereby incorporated by reference herein.

The preferred embodiments provide a continuous analyte sensor that measures a concentration of the analyte of interest or a substance indicative of the concentration or presence of the analyte. In some embodiments, the analyte sensor is an invasive, minimally invasive, or non-invasive device, for example a subcutaneous, transdermal, intravascular, or extracorporeal device. In some embodiments, the analyte sensor may analyze a plurality of intermittent biological samples. The analyte sensor may use any method of analyte-measurement, including enzymatic, chemical, physical, electrochemical, spectrophotometric, polarimetric, calorimetric, radiometric, or the like.

In some embodiments the analyte sensor may be broadly characterized as a diffusion-based sensor. Some particular embodiments of the diffusion-based sensor may be, more specifically, an electrochemical or electrode-based sensor. In some embodiments the electrochemical or electrode-based sensor may be an enzymatic sensor such as a GOX-based sensor or a GOX-based H₂O₂ sensor.

In general, analyte sensors provide at least one working electrode and at least one reference electrode, which are configured to measure a signal associated with a concentration of the analyte in the host, such as described in more detail below, and as appreciated by one skilled in the art. The output signal is typically a raw data stream that is used to provide a useful value of the measured analyte concentration in a host to the patient or doctor, for example. However, the analyte sensors of some embodiments comprise at least one additional working electrode configured to measure at least one additional signal, as discussed elsewhere herein. For example, in some embodiments, the additional signal is associated with the baseline and/or sensitivity of the analyte sensor, thereby enabling monitoring of baseline and/or sensitivity changes that may occur in a continuous analyte sensor over time.

In general, continuous analyte sensors define a relationship between sensor-generated measurements (for example, current in pA, nA, or digital counts after A/D conversion) and a reference measurement (for example, glucose concentration mg/dL or mmol/L) that are meaningful to a user (for example, patient or doctor). In the case of an implantable diffusion-based glucose oxidase electrochemical glucose sensor, the sensing mechanism generally depends on phenomena that are linear with glucose concentration, for example: (1) diffusion of glucose through a membrane system (for example, biointerface membrane and membrane system) situated between implantation site and/or the electrode surface, (2) an enzymatic reaction within the membrane system, and (3) diffusion of the H₂O₂ to the sensor. Because of this linearity, calibration of the sensor can be understood by solving an equation:

y=mx+b

where y represents the sensor signal (e.g., counts), x represents the estimated glucose concentration (e.g., mg/dL), m represents the sensor sensitivity to glucose (e.g., counts/mg/dL), and b represents the baseline signal (e.g., counts). When both sensitivity m and baseline (background) b change over time in vivo, calibration has generally requires at least two independent, matched data pairs (x₁, y₁; x₂, y₂) to solve for m and b and thus allow glucose estimation when only the sensor signal, y is available. Matched data pairs can be created by matching reference data (for example, one or more reference glucose data points from a blood glucose meter, or the like) with substantially time corresponding sensor data (for example, one or more glucose sensor data points) to provide one or more matched data pairs, such as described in U.S. Patent Publication No. US-2005-0027463-A1. In some implantable glucose sensors, such as described in more detail in U.S. Pat. No. 6,329,161 to Heller et al., which is incorporated herein by reference in its entirety, the sensing layer utilizes immobilized mediators (e.g., redox compounds) to electrically connect the enzyme to the working electrode, rather than using a diffusional mediator. In some implantable glucose sensors, such as described in more detail in U.S. Pat. No. 4,703,756, the system has two oxygen sensors situated in an oxygen-permeable housing, one sensor being unaltered and the other contacting glucose oxidase allowing for differential measurement of oxygen content in bodily fluids or tissues indicative of glucose levels. A variety of systems and methods of measuring glucose in a host are known, all of which may benefit from some embodiments to provide a sensor having a signal-to-noise ratio that is not substantially affected by non-constant noise. Additional description of analyte sensor configurations can be found in U.S. patent application Ser. No. 11/692,154, filed on Mar. 27, 2007 and entitled “DUAL ELECTRODE SYSTEM FOR A CONTINUOUS ANALYTE SENSOR”, U.S. Patent Publication No. US-2007-0027385-A1, and U.S. Patent Publication No. US-2005-0143635-A1.

Generally, implantable sensors measure a signal related to an analyte of interest in a host. For example, an electrochemical sensor can measure glucose, creatinine, or urea in a host, such as an animal (e.g., a human). Generally, the signal is converted mathematically to a numeric value indicative of analyte status, such as analyte concentration. It is not unusual for a sensor to experience a certain level of noise. In general, “constant noise” (sometimes referred to as constant background or baseline) is caused by non-analyte-related factors that are relatively stable over time, including but not limited to electroactive species that arise from generally constant (e.g., daily) metabolic processes. Constant noise can vary widely between hosts. In contrast, “non-constant noise” (sometimes referred to as non-constant background) is caused by non-constant, non-analyte-related species (e.g., non-constant noise-causing electroactive species) that arise during transient events, such as during host metabolic processes (e.g., wound healing or in response to an illness), or due to ingestion of certain compounds (e.g., certain drugs). In some circumstances, noise can be caused by a variety of noise-causing electroactive species.

In general, noise can be caused by a variety of factors, ranging from mechanical factors to biological factors. For example, macro- or micro-motion, ischemia, pH changes, temperature changes, pressure, stress, or even unknown mechanical, electrical, and/or biochemical sources can cause noise, in some embodiments. Interfering species, which cause non-constant noise, can be compounds, such as drugs that have been administered to the host, or intermittently produced products of various host metabolic processes. Exemplary interferents include but are not limited to a variety of drugs (e.g., acetaminophen), H₂O₂ from exterior sources (e.g., produced outside the sensor membrane system), and reactive metabolic species (e.g., reactive oxygen and nitrogen species, some hormones, etc.). Some known interfering species for a glucose sensor include but are not limited to acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid. In some cases noise may also arise when hosts are intermittently sedentary, such as during sleep or sitting for extended periods. When the host began moving again, the noise may quickly dissipate.

Noise is clinically important because it can induce error and can reduce sensor performance, such as by providing a signal that causes the analyte concentration to appear higher or lower than the actual analyte concentration. For example, upward or high noise (e.g., noise that causes the signal to increase) can cause the host's glucose concentration to appear higher than it truly is, which can lead to improper treatment decisions. Similarly, downward or low noise (e.g., noise that causes the signal to decrease) can cause the host's glucose concentration to appear lower than it is, which can also lead to improper treatment decisions. Accordingly, analyte sensor systems that are able to reduce noise arising in the analyte sensor offer important technological advantages.

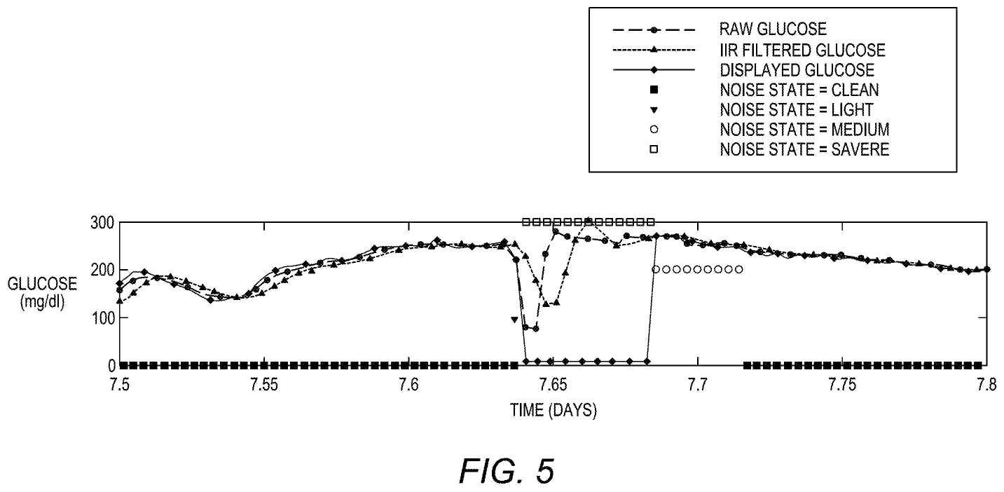

Conventional techniques for filtering the raw sensor signal may not always lead to satisfactory results. For instance, FIG. 5 shows the glucose level of a patient over a period of time as provided by a CGM system before the raw sensor signal is filtered. The raw sensor signal becomes significantly noisy shortly before time 7.65 and stays noisy past time 7.65. The noisy data may arise from a variety of sources, including, by way of example, displacement of the sensor in the patient due to the patient's movement or electronic error. The figure also shows the signal after being filtered using a conventional IIR filter. However, the filtered signal clearly does not accurately track the signal from the sensor for some time after the noisy data is received. Accordingly, the data may not be presented to the user for an extended period of time. FIG. 5 also shows that the data displayed to the user and the near zero value for the glucose level during this period indicates that no data is displayed for this entire period of time.

The Kalman filter belongs to the class of Bayesian estimators, which are a group of algorithms that extract information about a set of unknown variables or states given noisy measurements and some prior knowledge about the variables. Kalman filtering may use a two-step estimation process to extract information about the unknown variables by assuming that they are represented by probability density functions rather discrete values. Additional details of the Kalman filter estimation process generally may be found in S. Akhlaghi, N. Zhou and Z. Huang, “Adaptive adjustment of noise covariance in Kalman filter for dynamic state estimation,” 2017 IEEE Power & Energy Society General Meeting, Chicago, Ill., 2017, pp. 1-5 (“Akhlaghi”), which is hereby incorporated by reference in its entirety. This estimation process can be applied to continuous glucose monitor (CGM) measurements as described below.

In one example, as applied to analyte (e.g., CGM) measurements, the Kalman filter processes the raw analyte (e.g., glucose) signal (the noisy measurements) from the CGM sensor and provides an estimation of the filtered analyte (e.g., glucose) signal (the first unknown variable) by removing the noise from the raw analyte signal. It also provides a rough estimate of the analyte (e.g., glucose) signal rate of change (the second unknown variable).

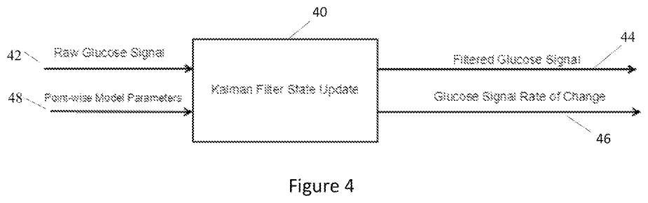

FIG. 4 is a simplified block diagram showing exemplary primary inputs to and outputs from the Kalman filter module 40. The inputs include a raw glucose signal 42 and point-wise model parameters 48. The raw glucose signal 42 represents the glucose signal values obtained from the CGM sensor, which may typically be provided at regular intervals of time (e.g., every 30 seconds, every 5 minutes, etc.). The point-wise model parameters 48 may be used to convert the glucose signal values (typically measured in units of pa) to glucose values (typically measured in units of mg/dl).

The outputs from the Kalman filter module 40 may be a filtered glucose signal 44 and a glucose signal rate of change 46. The filtered glucose signal 44 may be an estimation of the denoised glucose signal. The glucose signal rate of change 46 may be used in subsequent modules to estimate a trend value and/or other information or analytics.

The Kalman filter may perform an iterative (e.g., two-step) estimation process in which a predicted estimate of the filtered glucose signal and its rate of change is first determined (referred to as the a priori estimate), followed by a correction step in which the predicted estimate of the filtered glucose signal is updated.

The operation of the Kalman filter may be based on a state space model where:

$${x}_{k} = [ \begin{matrix}{g}_{k} \\ {d}_{k}\end{matrix} ]$$

In one exemplary embodiment, xₖ is the unknown state variable, gₖ is the unknown glucose signal value at time k, and dₖ is the unknown rate of change of the glucose signal at time k.

The state space model may define how, at each time k, the unknown variables in the state space model can be predicted from the previous step k, which may be given by:

$${x}_{k} = F \times {x}_{k - 1} + {w}_{k - 1}$$

$$where$$

$$F = [ \begin{matrix}1 & {Δ}_{t} \\ 0 & 1\end{matrix} ]$$

Δₜ indicates the time difference between the two iteration steps (e.g., the sampling time of the raw glucose signal) and which, for instance, may be equal to 0.5 minutes if the CGM sensor provides raw glucose signal values at 30-second intervals. The time difference and/or sample rate may be chosen to be any suitable time difference or sample rate. In some cases, the time difference and/or sample rate may be a dynamic and/or adaptive time difference or sample rate. wₖ₋₁ is the state process noise, where the mean may be equal to zero and the covariance matrix of the process noise at time k may be assumed to be given by Qₖ=E(wₖwₖ^{T}) under the assumption that the process noise has a multivariate normal distribution.

The measurement model determines how the unknown (state) variable xₖ is related to the observed or measured value yₖ (e.g., the raw glucose signal from the CGM sensor), which may be given by:

yₖ=H×xₖ+vₖ

where
H=[1 0].
   vₖ is the measurement noise, where the mean is equal to zero and the covariance matrix of the measurement noise at time k is assumed to be given by Rₖ=E(vₖvₖ^{T}) under the assumption that the measurement noise has a multivariate normal distribution.

Given the above definitions, in each iteration of the Kalman filter process, the two steps of prediction and correction may be performed as discussed below.

In the prediction step, an a priori estimate of the unknown state variable xₖ is obtained based on knowledge of the state variable at k−1 and the state model. In particular, the a priori estimate is

\hat{x}ₖ=F\hat{x}ₖ₋₁⁺

Pₖ⁻=FPₖ₋₁⁺F^{T}+Qₖ₋₁

where the superscript “+” indicates that the estimate is a posteriori and “−” indicates the estimate is a priori, and it is referenced with respect to the current observation at time k.

In the correction step, which may occur after the prediction step, the a priori estimate of the state variable xₖ is revised to obtain a more accurate estimate, which is referred to as the a posteriori estimate. Specifically, the a posteriori estimate of the state variable xₖ is calculated using the a priori estimate of the state variable xₖ, the current noisy measured value yₖ and the measurement equation. That is, the prediction step determines the value of the state variable xₖ before considering the measured value yₖ. The correction step then revises the value of the state variable xₖ by taking into account the measured values at time k. The detailed calculation is given below:

dₖ=yₖ−H \hat{x}ₖ⁻

Pᵢₙₙₒᵥ=HPₖ⁻H^{T}+Rₖ

Gₖ=Pₖ⁻H^{T}[Pᵢₙₙₒᵥ]⁻¹

\hat{x}ₖ⁺=\hat{x}ₖ+Gₖdₖ

Pₖ⁺=Pₖ⁻−GₖPᵢₙₙₒᵥGₖ^{T}

where dₖ is the innovation term and Pᵢₙₙₒᵥ is the innovation covariance. The Kalman gain is indicated by Gₖ. The a posteriori estimate for the state variable and covariance matrix is given by \hat{x}ₖ⁺ and Pₖ⁺ respectively.

Based on the above equations, the updated a posteriori state estimate can be calculated in each Kalman filter iteration step. The additional values to be determined are the initial values for the x₀⁺ and Pₖ⁺, which may be provided during an initialization step.

In one embodiment, the application of the Kalman filter to raw glucose signals from a CGM sensor may be summarized as follows. If, for example, a CGM sensor generates a measured value every e.g., 30 seconds, then a count or sample is received by the Kalman filter every 30 seconds. Assume at a time t=150 sec that a count is received and at this time the Kalman filter, in the prediction step, predicts what the state variable xₖ will be based on the counts received up to and including t=120 sec. The prediction is based on the previously obtained measured counts obtained from the sensor and the assumptions employed by the state model about how glucose levels change over time. Next, the correction step is performed at t=150 sec where the estimate of the state variable is updated using the most recent measured count value. Thus, at t=150 sec there is a predicted value of the unknown state variable x available and a measured value y. The error in the predicted value is obtained by comparing these two values. This error is referred to as the measurement innovation since it is the new information that is obtained based on the observation or measurement at time k. Other embodiments may use other time intervals for generation of a measured value and/or operation of Kalman filter processing (e.g., every 15 seconds, 1 minute, 5 minutes, etc.).

Two noise components may be employed in the Kalman filter, the process noise and the measurement noise. These noise components may be known in advance and/or estimated from the data. The measurement noise may approximately correspond to the noise present on the observed signal and the process noise may approximately correspond to the model error. The correct estimation of these noise components may have an impact on the performance of the Kalman filter in terms of the optimal removal of noise and/or its robustness when a signal anomaly arises. The measurement innovation described above may be used to update the measurement covariance R and the process covariance Q. The updated values of Q and R can then be used to update other parameters used by the Kalman filter, such as the Kalman gain and/or the a posteriori state values.

A conventional Kalman filtering process may not produce a high-quality filtered signal when certain underlying assumptions about noise (e.g., its Gaussian nature) is violated. This filtering process may result in a relatively long period of down time when no glucose values are displayed to the user. These problems may be addressed by the techniques described below, which modify the process of updating the noise covariance terms Q and R. Various embodiments may be employed for this purpose, as listed below and subsequently explained in more detail:
Estimating the values for Q and R using innovation and residual error values in each step.
Estimating the values for Q and R using innovation and residual error values in each step using adjustable adaptation coefficients that are calculated based on the possibility of the presence of a signal anomaly.
Modifying the Kalman filter estimation step if a signal anomaly is detected.
Adding an artifact detection module to detect signal anomalies.

As noted above, these innovation and residual error values may be used to estimate the values of Q and R, for example by adaptively adjusting their values, either using constant coefficients or using data-driven features to adjust the adaptation coefficients. This may enable the Kalman filter to be more robust to signal anomalies and/or attain a better tradeoff in terms of removing noise and tracking signal changes with less lag.

In some embodiments, the process and measurement noise terms may be updated differently when certain artifacts are identified. The manner in which such artifacts are identified or otherwise determined to be present may differ in different implementations. For instance, in some embodiments, discussed in more detail below, such artifacts may be identified by examining certain features in the sensor signal. In yet other embodiments, also discussed below, an indication of the presence of such artifacts may be determined by examining one or more metrics based on internal variables used in the Kalman filter.

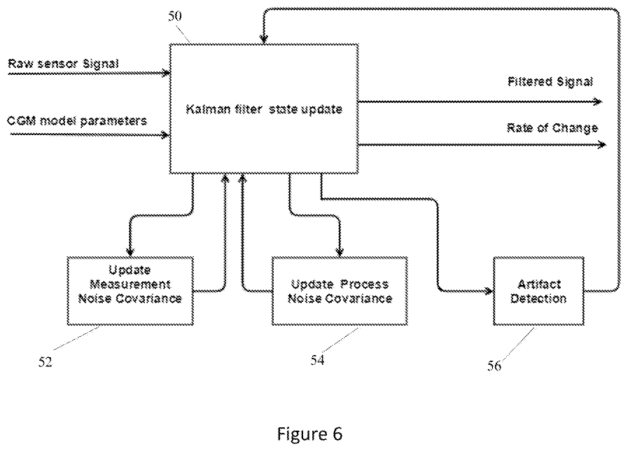

FIG. 6 shows a simplified block diagram of one example of a Kalman filter in which an artifact detection module 56 is employed to examine the sensor signal output by the Kalman filter state update module 50. Artifact detection may be performed after the Kalman filter updates the sensor signal to detect the presence of signal anomalies on the sensor signal. The exemplary Kalman filter of FIG. 6 also includes a measurement noise covariance module 52 and a process noise covariance module 54, which provide updated values of the measurement noise covariance matrix and the process noise covariance matrix, respectively. If an artifact is detected by the artifact detection module 56 certain preventive and/or corrective actions may be taken regarding the updates to the measurement noise covariance matrix and the process noise covariance matrix, as discussed in more detail below.

In some embodiments, the artifact detection module examines the residual signal, which is defined as the difference between the raw sensor signal and the estimated (filtered) sensor signal after being updated by the Kalman filter. The residual signal may be defined in two different steps. In a first step, a temporary residual signal may be defined before updating the measurement covariance R, the process covariance Q and the other parameters such as the Kalman gain G. In the second step, a final residual signal may be defined after updating the measurement covariance R, the process covariance Q and the other parameters such as the Kalman gain G. Various features of the temporary and/or final residual signals may be indicative of artifacts that may result in certain preventive and/or corrective actions being taken regarding the updates to the internal variables in the Kalman filter such as the state variable and/or noise covariances. In general, features indicative of signal artifacts may be extracted from either or both of the residual signals (temporary and final) and/or from the interaction or relationship between the two residual signals.

For instance, one feature that may be indicative of an artifact is the residual difference, which is defined as the difference between the value of the temporary residual signal (the residual signal before updating the Kalman parameters) and the value of the final residual signal (the residual signal after updating the Kalman parameters). The residual difference (or a derivative of the residual difference) may be compared to a predefined threshold such as a data-driven predefined threshold in the residual signal domain. A signal artifact may be present if the residual difference is above (or below) the threshold. In one alternative embodiment, the residual difference may be translated to a corresponding difference in the estimated glucose value by applying the necessary model parameters used to perform the translation or calibration. In this way the residual difference in the glucose domain can be compared to a predefined threshold in order to detect the presence of signal artifacts. In general, different mathematical operations can be applied to the residual difference in the signal domain or in the glucose value domain in order to identify signal artifacts.

Another feature that may be examined for the presence of an artifact is the residual bias, which determines if there are consistent high magnitude positive or negative values in the final residual signal over different time windows. In this context, the final residual signal is defined as the smoothed value of the difference between the raw sensor signal and the estimated sensor signal output by the Kalman filter. The accumulation of negative or positive final residual values in a given window of time may suggest that the assumption that the noise is white Gaussian noise is not valid. In this way the residual bias may serve to indicate the presence of an artifact.

Yet another feature that may be examined for the presence of an artifact is referred to as the zero crossing of the final residual signal. This feature may track the number of sign changes in the final residual signal over different time windows. In this context, the final residual may be defined as smoothed value of the difference between the raw sensor signal and the estimated signal output by the Kalman filter. A large number of zero crossings may indicate the presence of unbiased noise, whereas a smaller number of zero crossings may indicate biased noise and hence the presence of artifacts.

By identifying artifacts, the residual bias and/or zero crossing features can be used to identify the unreliable portions of the signal so that preventive and/or corrective action is taken, which will be discussed in greater detail below. These features also can be applied retroactively to the past history of the signal to improve the performance of the system. In addition, the residual bias can be used not only to detect the presence of an artifact, but also to detect the presence of a step anomaly, which may occur, for instance, when pressure is suddenly applied to the sensor such as when the user lies down.

Other features may be examined for the presence of artifacts in alternative embodiments or examples. For instance, features that may be used for real time artifact detection are model-based change measures, including a median/mean model that is subtracted from the signal, linear models over time that are subtracted from the signal, innovation value, residual value, the sign of the innovation/residual, R/Q value, and/or the residual kurtosis.

Once an artifact is detected, a rule-based model may be used to determine whether the feature should be classified as an artifact that should cause the process covariance and the measurement covariance to be updated and/or to cause other actions to be taken. For instance, a data-driven decision tree model may be trained using clinical data to detect artifacts using any of the aforementioned features. Likewise, a wide variety of machine learning models may be applied to the above features, or a combination of features, to determine that an artifact is present.

For instance, in one implementation the preventive action that is taken upon detecting an artifact may undo the latest Kalman filter parameter update and maintain their values within a normal range. If corrective action is triggered several strategies can be followed depending, for instance, on the sampling frequency of the sensor signal. For example, in the case of low-resolution signal availability (i.e., a signal sampled at a relatively low frequency), different corrective action is triggered based on the sign of the signal residual. In the case of high-resolution signal availability (i.e., a signal sampled at a relatively high frequency), additional features such as the residual bias and the zero crossing (as described above) may be used retroactively to determine the optimal Kalman filter model that were used in the past as determined from the relevant historical data. In general, the corrective action that may be taken when updating the Kalman filter parameters to select their optimal values involves a tradeoff between the amount of signal smoothing (the amount of noise removed from the signal) and a lag in tracking the changes in the signal.

As previously mentioned, instead of and/or in addition to examining the sensor signal for artifacts after being processed by the Kalman filter, in other embodiments an indication of the presence of such artifacts may be determined by examining one or more metrics based on internal variables used in the Kalman filter.

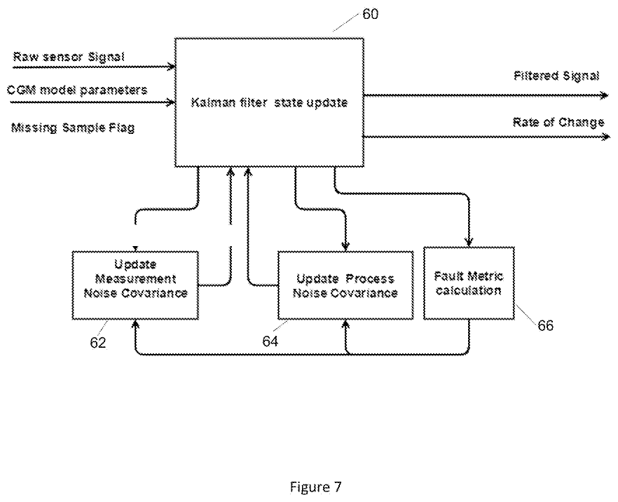

FIG. 7 shows a simplified block diagram of one example of a Kalman filter in which a fault metric calculation module 66 is employed to examine various internal variables used by the Kalman filter update module 60. The exemplary Kalman filter of FIG. 7 also includes a measurement noise covariance module 62 and a process noise covariance module 64, which provide updated values of the measurement noise covariance matrix and the process noise covariance matrix, respectively, based on the value of the fault metric that is received from the fault metric calculation module.

In one embodiment, the fault metric that is employed may be based on the fault metric discussed in Zheng et al., A Robust Adaptive Unscented Kalman Filter for Nonlinear Estimation with Uncertain Noise Covariance. Sensors 2018, 18, 808. In particular, the fault metric may be defined as the moving average of a temporary or instantaneous fault metric averaged over a specified number (e.g., 10) of measurement samples. More specifically, the temporary fault metric may be given by:

temporary fault metric=dₖ^{T}[Pᵢₙₙₒᵥ]⁻¹dₖ

where dₖ is the innovation term and Pᵢₙₙₒᵥ is the innovation covariance. The temporary fault metric may be the normalized innovation squared and the fault metric is a moving average of this term. High values of the fault metric may indicate that a signal anomaly has occurred and therefore, it can be used to readjust the Kalman filter parameters for the affected data points.

Examples of how the measurement noise covariance matrix and the process noise covariance matrix may be updated based on the value of the fault metric are presented below.

In one embodiment, the covariance matrix of the measurement noise (Rₖ) may be adaptively updated in each iteration (k) of the Kalman filter based on the residual signal (εₖ) and a step size (αᵣ), given by:

Rₖ=αᵣ(εₖεₖ^{T}+H Pₖ⁻H^{T})+(1−α_{R})Rₖ₋₁

where

εₖ=yₖ−Hxₖ⁺

Pₖ⁻: a priori estimate covariance
H: observation model
yₖ: observed signal
xₖ⁺: aposteriori state estimate.

Likewise, in one embodiment the covariance matrix of the process noise may be given by

Qₖ=α_{q}(Gₖdₖdₖ^{T}Gₖ^{T})+(1−α_{q})Qₖ₋₁

where

dₖ=yₖ−H\hat{x}ₖ⁻

Gₖ: Kalman gain
\hat{x}k̅: a priori state estimate. b

In some implementations the covariance matrix Qₖ of the process noise is a 2×2 matrix, where the element Qₖ (2,2) controls the changes to the estimated rate of change of the signal. Smaller values of Qₖ (2,2) may result in slower changes to the estimated model and therefore more smoothing of the sensor signal. On the other hand, higher values of Qₖ (2,2) may result in faster changes to the estimated model and therefore more tracking of the sensor signal. A minimum value may be applied to the term in covariance matrix Qₖ i.e., if the Qₖ (2,2) is smaller than Qₘᵢₙ value, it may be capped to be equal to the Qₘᵢₙ. In some implementations the minimum value (Qₘᵢₙ) may be a constant value.

In one embodiment, after the fault metric is calculated, the step size coefficients used in updating the measurement and process noise covariances (αᵣ, α_{q}) and the applied minimum value (Qₘᵢₙ) may be adjusted using the following transfer functions based on the fault metric ƒₖ as follows:

$${α}_{r} = max ( {α}_{r}^{default} , \frac{fk - {α}_{r}^{InitPoint}}{{f}_{k}} )$$

$${α}_{q} = 1 - max ( {α}_{q}^{default} , \frac{fk - {α}_{q}^{InitPoint}}{{f}_{k}} )$$

$${Q}_{min} = max ( \frac{{Q}_{min}^{max}}{1 + e ( {f}_{k} - {Q}_{min}^{FMPoint} )} , {Q}_{min}^{default} )$$

The design parameters such as αᵣ^{default}, αᵣ^{InitPoint}, αᵣ^{InitPoint}, α_{q}^{InitPoint}, Qₘᵢₙ^{max}, Qₘᵢₙ^{FMPoint} and Qₘᵢₙ^{default} may be optimized based on population data to achieved the desired trade-off between smoothing and time lag at areas with high rate of change.

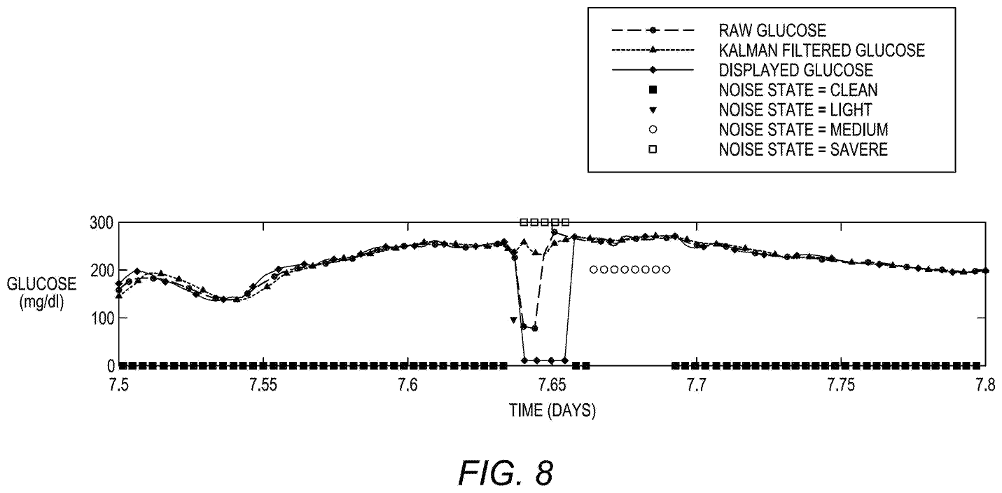

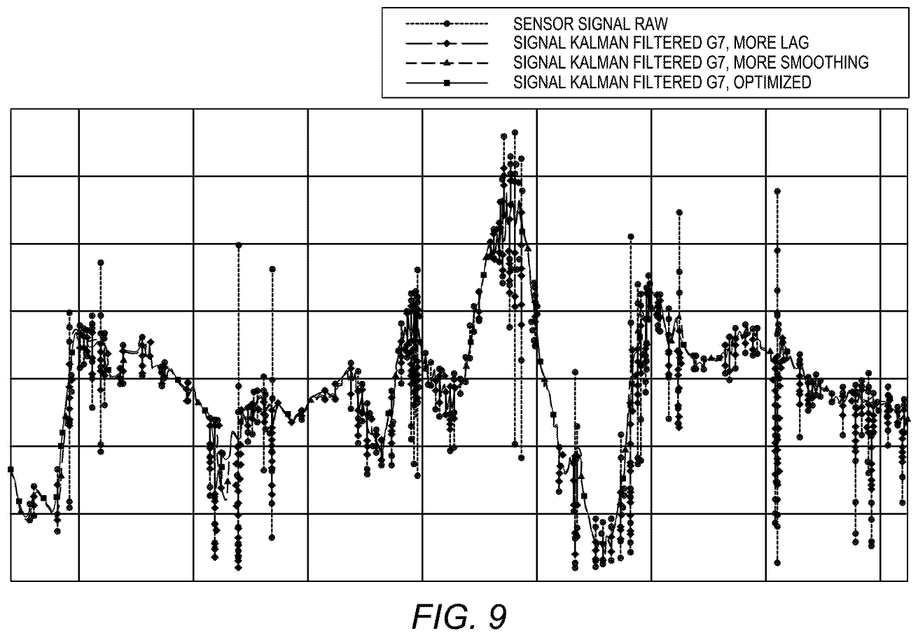

FIG. 8 shows the same raw sensor signal shown in FIG. 5, except in FIG. 8 the signal is filtered using a Kalman filter configured in accordance with at least some of the techniques described herein. As shown, the filtered signal allows data to be continuously presented to the user. Having no or reduced periods of time during which no data is presented to the user may represent an improvement over the filtered signal shown in FIG. 5. FIG. 9 shows another raw sensor signal and filter sensor signal after being filtered with a Kalman filter using three different sets of parameters. One curve represents the filtered signal when the set of parameters is adjusted to provide more smoothing. Another curve represents the filtered signal when the set of parameters is adjusted to provide a greater time lag. A third filtered signal represents the filtered signal when the set of parameters is adjusted to provide an overall level of optimization.

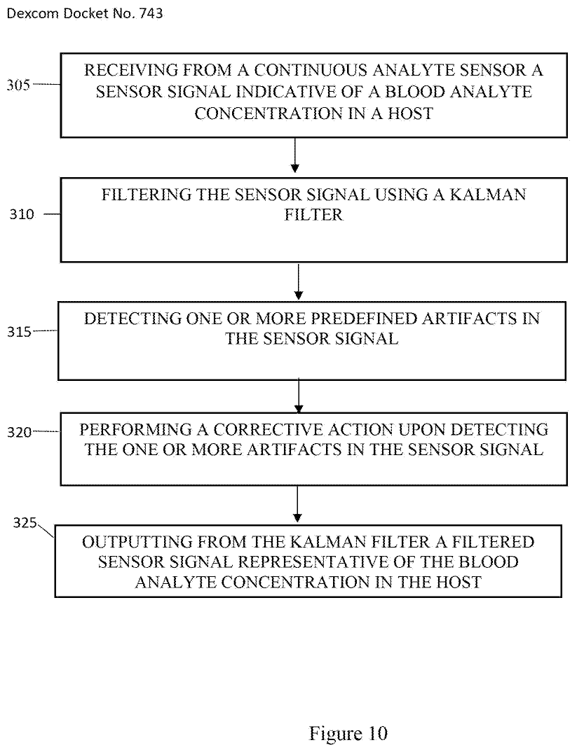

FIG. 10 is an exemplary flowchart showing one example of a method for monitoring a blood analyte concentration in a host. In accordance with the method, a sensor signal indicative of a blood analyte concentration in a host is received from a continuous analyte sensor at step 305. At step 310 the sensor signal is filtered using a Kalman filter. One or more artifacts (e.g., predefined) is detected in the sensor signal at step 315. At step 320, a corrective action is performed upon detecting the one or more artifacts in the sensor signal. The corrective action may include updating values associated with one or more of parameters employed in a model of the Kalman filter. A filtered sensor signal representative of the blood analyte concentration in the host is output from the Kalman filter at step 325. In an alternative embodiment, additional, fewer, and/or different steps and/or differing ordering of steps may be performed than those explicitly shown for FIG. 10.

The various operations of methods described above may be performed by any suitable means capable of performing the operations, such as various hardware and/or software component(s), circuits, and/or module(s). Generally, any operations illustrated in the figures may be performed by corresponding functional means capable of performing the operations. Notably, use of the term “module” does not limit functionality performed by a given module to a separate and discrete module. Instead, functionality described as being performed by a given module may also be performed by a system executing on a single processor even if the functionality is not separated into discrete modules.

The various illustrative logical blocks, modules and circuits described in connection with the present disclosure may be implemented or performed with a general purpose processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described herein. A general purpose processor may be a microprocessor, but in the alternative, the processor may be any commercially available processor, controller, microcontroller or state machine. A processor may also be implemented as a combination of computing devices, e.g., a combination of a DSP and a microprocessor, a plurality of microprocessors, one or more microprocessors in conjunction with a DSP core, or any other such configuration.

In one or more aspects, the functions described may be implemented in hardware, software, firmware, or any combination thereof. If implemented in software, the functions may be stored on or transmitted over as one or more instructions or code on non-transitory computer-readable medium. By way of example, and not a limitation, such non-transitory computer-readable media can comprise RAM, ROM, EEPROM, CD-ROM or other optical disk storage, magnetic disk storage or other magnetic storage devices.

The methods disclosed herein comprise one or more steps or actions for achieving the described methods. The method steps and/or actions may be interchanged with one another without departing from the scope of the claims. In other words, unless a specific order of steps or actions is specified, the order and/or use of specific steps and/or actions may be modified without departing from the scope of the claims.

Certain aspects may comprise a computer program product for performing the operations presented herein. For example, such a computer program product may comprise a computer readable medium having instructions stored (and/or encoded) thereon, the instructions being executable by one or more processors to perform the operations described herein. For certain aspects, the computer program product may include packaging material.

Software or instructions may also be transmitted over a transmission medium. For example, if the software is transmitted from a web site, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes and variations may be made in the arrangement, operation and details of the methods and apparatus described above without departing from the scope of the claims.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as and/or unless expressly stated otherwise. Similarly, a group of items linked with the conjunction ‘or’ should not be read as requiring mutual exclusivity among that group, but rather should be read as and/of unless expressly stated otherwise.

Where a range of values is provided, it is understood that the upper and lower limit and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention, e.g., as including any combination of the listed items, including single members (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

All references cited herein are incorporated herein by reference in their entirety. To the extent publications and patents or patent applications incorporated by reference contradict the disclosure contained in the specification, the specification is intended to supersede and/or take precedence over any such contradictory material.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method for monitoring an analyte concentration, the method comprising:
receiving, from an analyte sensor, a sensor signal indicative of an analyte concentration in a host;
filtering the sensor signal using a Kalman filter having process noise with a process covariance and measurement noise with a measurement covariance, wherein the filtering includes updating a value of at least one of the process covariance or the measurement covariance using a value of one or more parameters employed in a model of the Kalman filter; and
outputting, from the Kalman filter, a filtered sensor signal representative of the analyte concentration in the host.

2. The method of claim 1, wherein the one or more parameters used to update at least one of the process covariance and the measurement covariance includes a value of an innovation term and a residual term employed in the Kalman filter model.

3. The method of claim 1, wherein the updating is performed when one or more predefined artifacts are detected in the sensor signal.

4. The method of claim 3, further comprising detecting the one or more predefined artifacts by examining a residual signal, the residual signal being a difference between the sensor signal received from the analyte sensor and the sensor signal after filtering the sensor signal using the Kalman filter.

5. The method of claim 4, wherein the residual signal is a temporary residual signal that is a difference between the sensor signal received from the analyte sensor and the sensor signal after filtering the sensor signal using the filter before the at least one of the process covariance and the measurement covariance is updated.

6. The method of claim 4, wherein the residual signal is a final residual signal that is a difference between the sensor signal received from the analyte sensor and the sensor signal after filtering the sensor signal using the filter after the at least one of the process covariance and the measurement covariance is updated.

7. The method of claim 4, wherein one of the predefined artifacts is a residual bias reflecting that the residual signal has a consistently positive or negative value over one or more selected windows of time.

8. The method of claim 7, wherein one of the predefined artifacts is a zero crossing of a final residual signal, the zero crossing of the final residual signal reflecting a number of times a value of the final residual signal undergoes a change in sign from positive to negative or negative to positive over one or more selected windows of time.

9. The method of claim 1, further comprising undoing a previous update to the values of at least one of the process covariance and the measurement covariance upon detecting one or more specified artifacts in the sensor signal.

10. The method of claim 1, wherein the one or more parameters used to update at least one of the process covariance and the measurement covariance includes a fault metric that is based on a value of an innovation term and an innovation covariance employed in the Kalman filter model.

11. The method of claim 10, wherein the fault metric is a moving average of an instantaneous fault metric averaged over a specified number of measurement samples received from the analyte sensor.

12. The method of claim 1, further comprising performing a corrective action upon detecting one or more artifacts in the sensor signal when the sensor signal is a low-resolution signal, the corrective action being determined at least in part by a sign of a residual signal, the residual signal being a difference between the sensor signal received from the analyte sensor and the sensor signal after filtering the sensor signal using the Kalman filter.

13. The method of claim 1, further comprising retroactively determining from historical data an optimal Kalman filter model that was previously employed when the sensor signal is a high-resolution signal.

14. The method of claim 13, wherein the determining is performed using a residual bias and a zero crossing, the residual bias reflecting that a residual signal has a consistently positive or negative value over one or more selected windows of time and the zero crossing reflecting a number of times the residual signal undergoes a change in sign from positive to negative or negative to positive over one or more selected windows of time.

15. A method for monitoring an analyte concentration, the method comprising:
receiving from an analyte sensor a sensor signal indicative of an analyte concentration in a host;
filtering the sensor signal using a Kalman filter;
detecting one or more artifacts in the sensor signal;
performing a corrective action upon detecting the one or more artifacts in the sensor signal, wherein the corrective action includes updating values of one or more of parameters employed in a model of the Kalman filter; and
outputting, from the Kalman filter, a filtered sensor signal representative of the analyte concentration in the host.

16. The method of claim 15, wherein the detecting the one or more artifacts in the sensor signal comprises examining one or more internal variables of the Kalman filter to detect the artifact, wherein the one or more internal variables include a fault metric.

17. A method for monitoring an analyte concentration, the method comprising:
receiving, from an analyte sensor, a sensor signal indicative of an analyte concentration in a host;
filtering the sensor signal using a Kalman filter;
during the filtering, examining a residual signal to detect an artifact in the sensor signal, the residual signal comprising a difference between the sensor signal and an estimated filtered sensor signal generated by the Kalman filter; and
responsive to detecting the artifact in the sensor signal, updating the estimated filtered sensor signal.

18. The method of claim 17, wherein the artifact is detected based on a residual bias reflecting that the residual signal has a consistently positive or negative value over one or more time periods.

19. The method of claim 17, wherein the artifact is detected based on a zero crossing indicating a number of times the residual signal undergoes a change in sign over one or more time periods.

20. The method of claim 17, wherein the artifact is detected by comparing the residual signal to a predefined threshold.
